(19) United States
(12) Patent Application Publication
(10) Pub. No.: US 2021/0402022 A1
(43) Pub. Date: Dec. 30, 2021

(54) Ultraviolet light pod

(71) Applicant: Nemesis UVC LLC, Garland, TX (US)

(72) Inventors: Michael Nevitt, Garland, TX (US); Eric L. Wade, Garland, TX (US); John Early, Garland, TX (US)

(21) Appl. No.: 17/333,055

(22) Filed: May 28, 2021

Related U.S. Application Data
(60) Provisional application No. 63/032,199, filed on May 29, 2020.

(51) Int. Cl. A61L 2/10

(52) U.S. Cl. CPC A61L 2/10; A61L 2202/25; A61L 2202/14; A61L 2202/11

(57) ABSTRACT

Devices, systems, and methods for disinfecting and/or sanitizing an area using multiple ultraviolet (UV) lights or panels. The devices and systems may include a fixed or movable UV light pod configured to maximize the photometric overlay of ultraviolet (UV) light delivered to a surface or area. This UV light pod is configured to expose the surface/area to a maximum amount of UV light, thus decreasing the amount of UV light exposure time applied to the surface/area to properly sanitize the area or surface.

### CROSS REFERENCE TO RELATED APPLICATIONS

In accordance with 37 C.F.R. 1.76, a claim of priority is included in an Application Data Sheet filed concurrently herewith. Accordingly, the present invention claims priority to U.S. Provisional Patent Application No. 63/032,199, entitled “ULTRAVIOLET LIGHT POD” filed May 29, 2020. The contents of the above referenced application are incorporated herein by reference in their entirety.

### FIELD OF THE INVENTION

The invention relates to devices, systems, and methods for lighting and/or disinfecting an area; to devices and methods for housing one or more ultraviolet (UV) lights or panels to prevent microbial or pathogen growth within an area; and more particularly, to a portable ultraviolet (UV) light pod for use in preventing microbial or pathogen growth within an area.

### BACKGROUND OF THE INVENTION

The worldwide 2020 Coronavirus pandemic emergency illustrates the need for preventing or inhibiting microbial growth, such as the virus responsible for COVID 19, to prevent disease. The need to quickly and easily provide devices and systems as part of an overall strategy for preventing or minimizing all microbial growth in every aspect of society is more important than ever.

### SUMMARY OF THE INVENTION

Devices, systems, and methods for disinfecting an area using multiple ultraviolet (UV) lights or panels are described. The devices and systems include a fixed or movable UV light pod configured to maximize the photometric overlay of the UV light to a surface or area. This configuration allows the UV light pod to expose the surface/area to the maximum amount of UV light, thus decreasing the amount of UV light exposure time applied to the surface/area to properly sanitize the area or surface. Preferably, the UV light pod has a hexagonal shape or hexagonal cross-section.

Accordingly, it is an objective of the invention to provide a unique disinfecting and/or sanitizing device, system, and method.

It is a further objective of the invention to provide disinfecting and/or sanitizing devices, systems, and methods using UV light.

It is a still further objective of the invention to provide a UV light based device or system configured to disinfect and/or sanitize surfaces or areas.

It is a further objective of the invention to provide a UV light pod configured to disinfect and/or sanitize large surfaces or areas.

It is yet another objective of the invention to provide a hexagonal shaped UV light pod configured to disinfect and/or sanitize large surfaces or areas.

It is a still further objective of the invention to provide a UV light pod configured to disinfect and/or sanitize large surfaces or areas.

It is a further objective of the invention to provide a UV light pod configured to provide a photometric overlay of UV light exposure to a surface.

It is yet another objective of the invention to provide a UV light pod configured to provide a photometric overlay of UV light to maximize surface/area exposure, thus decreasing the exposure time required to sanitize the area or surface.

It is a still further objective of the invention to provide a UV light pod configured to disinfect and/or sanitize large surfaces or areas in combination with one or more non-UV light sources configured to illuminate a space, surface, or area.

It is a still further objective of the invention to provide a UV light pod configured to disinfect and/or sanitize an area or surface which uses an in-use warning system to prevent unwanted UV light exposure.

It is yet another objective of the invention to provide a UV light pod configured to disinfect and/or sanitize an area or surface which uses an occupant detection sensor system to prevent unwanted UV light exposure.

Other objectives and advantages of this invention will become apparent from the following description taken in conjunction with any accompanying drawings wherein are set forth, by way of illustration and example, certain embodiments of this invention. Any drawings contained herein constitute a part of this specification, include exemplary embodiments of the present invention, and illustrate various objects and features thereof.

### BRIEF DESCRIPTION OF THE FIGURES

FIG. 1 is a perspective view of an illustrative embodiment of the UV light pod sanitization device;

FIG. 2 is a bottom view of the UV light pod sanitization device;

FIG. 3 is a top view of the UV light pod sanitization device;

FIG. 4 is a side view of the UV light pod sanitization device;

FIG. 5 illustrates the UV light pod sanitization device hanging from a ceiling;

FIG. 6 illustrates the UV light pod sanitization device with a dynamic hoist system;

FIG. 7 illustrates the 360 degree overlap of UV light beam angles;

FIG. 8 is an illustrative embodiment of the UV light pod having both UV light panels and non-UV light panels;

FIG. 9A is an illustrative embodiment of the UV light pod having UV light panels with UV LEDs;

FIG. 9B is an illustrative embodiment of the UV light pod having UV light panels with UV bulbs; and

FIG. 10 is an illustrative embodiment of the UV light pod placed within a stadium, hanging from the stadium ceiling.

### DETAILED DESCRIPTION OF THE INVENTION

While the present invention is susceptible of embodiment in various forms, there is shown in the drawings and will hereinafter be described a presently preferred, albeit not limiting, embodiment with the understanding that the present disclosure is to be considered an exemplification of the present invention and is not intended to limit the invention to the specific embodiments illustrated.

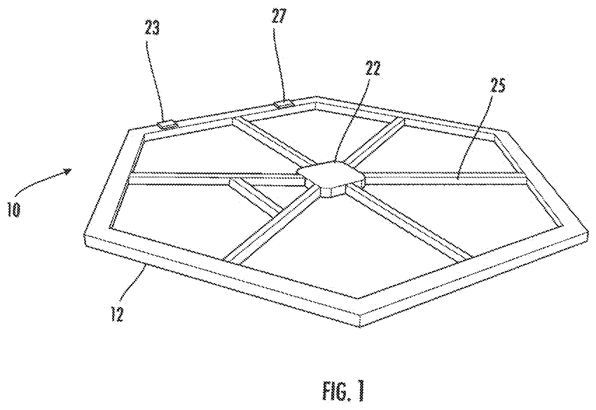

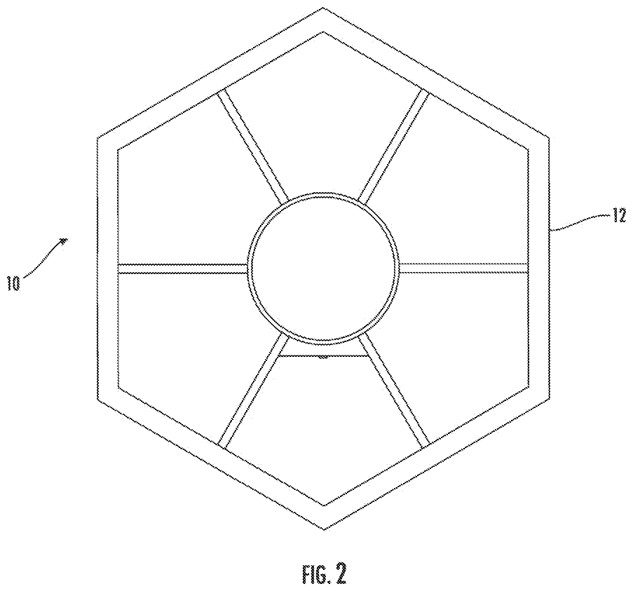

Referring to FIGS. 1-6, an illustrative embodiment of an ultraviolet (UV) light based sanitization device, referred to generally as a UV light pod 10, is shown. The UV light pod 10 is configured to maximize the photometric overlay of UV light applied to a surface or area to be disinfected and/or sanitized. Accordingly, the UV light pod 10 is designed to expose the surface/area to the maximum amount of UV light, thus decreasing the amount of UV light exposure time required to disinfect and/or sanitize the area or surface.

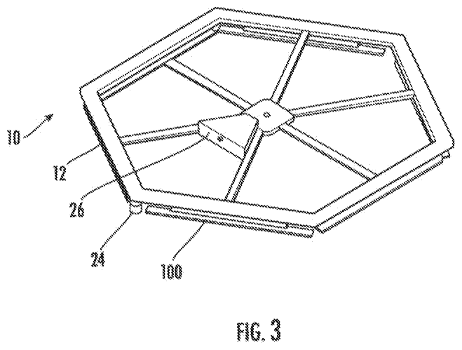

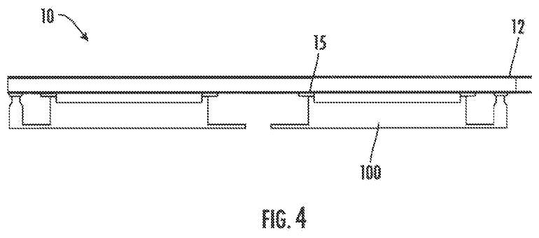

The UV light pod 10 comprises a support structure 12 sized and shaped to receive and secure thereto one or more sanitizing units, illustrated herein as UV light panels 100, see FIG. 3 or 4. Preferably, the support structure 12 comprises a hexagonal shape or cross sectional shape. Although the support structure 12 comprises a hexagonal shape or cross sectional shape, any shape that provides maximum UV light exposure may be used.

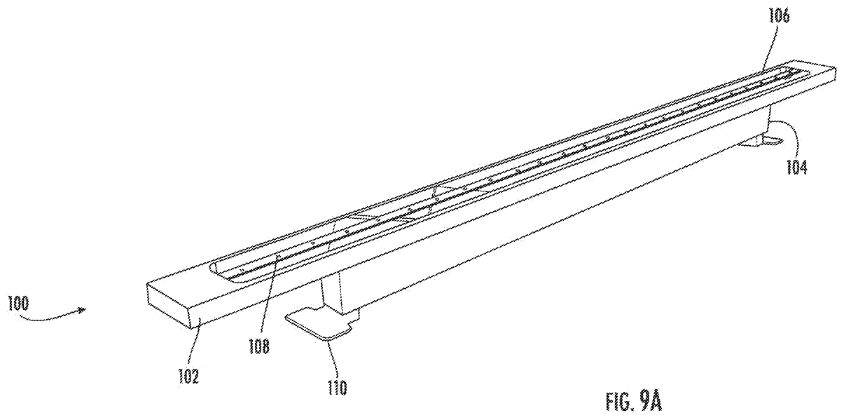

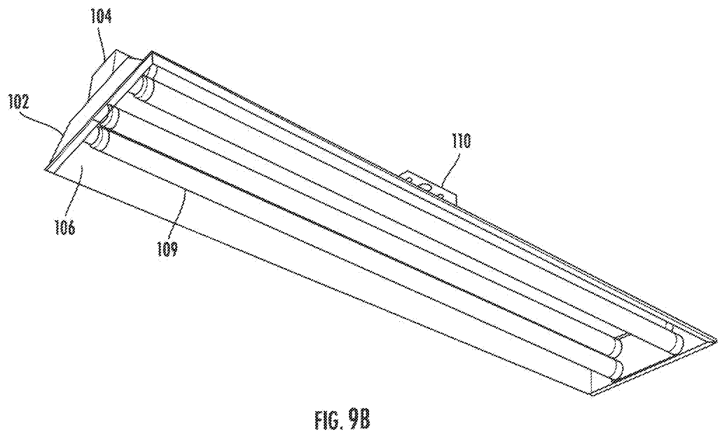

The UV light panel 100 may include a source of UV light, such as a standard UV light lamp or light emitting diodes (LEDs) UV lamp. The UV light panel 100 is preferably secured to the one or more portions of the UV light pod 10 via mechanical fastening members, such as screws and bolts, or brackets, and may be configured to pivot. FIG. 9A provides an illustrative example of the UV light panel 100. The UV light panel 100 may include a source housing 102, an electrical housing 104, which may include the electrical hardware and ballast (not shown), a reflector 106, an LED UV light source such as UV-LEDS 108 (but could be a radiator or lamp 109, see FIG. 9B) and a bracket 110 for securing to one or more portions of the UV light pod 10. Each individual UV light panel 100 may be configured to be separately removably mountable, such as via securing the bracket 110 to a support structure light bracket 15, see FIG. 4. Such functionality allows a user to add or remove the UV light panels 100 as needed.

The UV light panel 100 may include a power cord for plugging into an electrical outlet or an electrical outlet bank, or be operatively connected or wired, via connectors, for example, use of True One connectors/plugs (manufactured by Neutrik) or other mechanisms known to one of skill in the art, to multiple UV light panels 100 and a central or main power ON/OFF control unit or button (which may be on the unit or be part of a remotely located unit) so each of the multiple UV light panels 100 operate simultaneously when powered ON or powered OFF or timed run/operating cycles.

The UV light panel 100 includes a power cord (not shown) for plugging into an electrical outlet. Preferably, the UV light panel 100 utilizes one or more LED UV-C bulbs (emitting in the range of 200-400 nm, preferably in the range of approximately 254 nm to about 280 nm). As used herein, the term “about” or “approximately” defines any value between greater than zero and 20% above or below a stated value or range. While described as using UV-C light, other UV lights that have the ability to inactivate pathogens (both viruses and bacteria) by impacting the cellular RNA and DNA, damaging nucleic acids, and preventing microorganisms from infecting and reproducing, or acting as a disinfectant, may be used as well. The UV light pod 10 may be configured so that when powered ON (OFF), all UV light panels 100 turn ON (OFF) and emit light at the same time, or each individual UV light panel 100 may be turned ON (OFF) and emit light separately and independently.

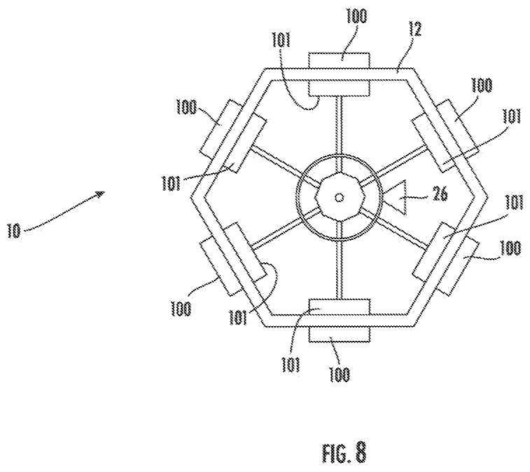

The UV light panel 100 may also include other non-UV light producing lamps, such as LED lights, and RGB LED 24/7 house or work lights for use in typical day to day illumination of the area or surface. Alternatively, or in combination with the UV light panel 100, the UV light pod 10 may include one or more separate, non-UV light producing illumination panels or lamps, such as LEDs. Referring to FIG. 8, an illustrative example of UV light pod 10 is shown with the support structure 12 having both UV light panels 100 and non-UV light panels 101.

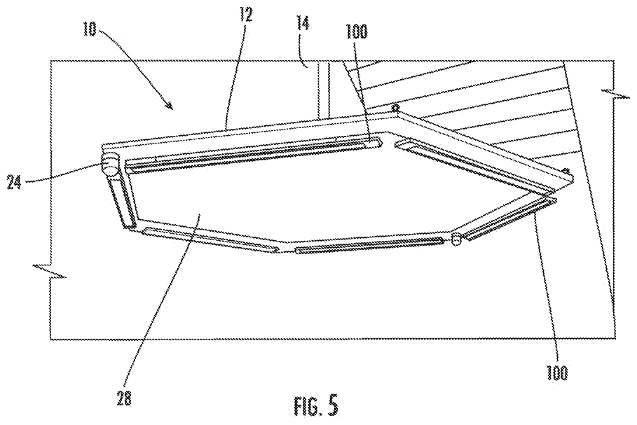

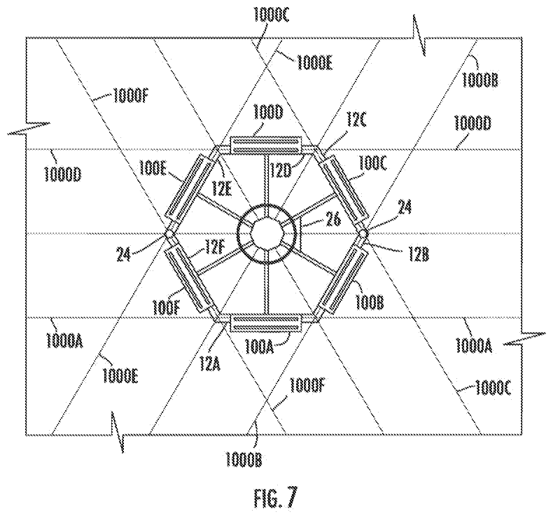

As illustrated in FIG. 3 or FIG. 5, the UV light pod 10 is configured to contain at least one UV light panel 100 secured to each of the portions or sections of the hexagonal shape of the support structure 12. This arrangement allows for a unique photometric UV light overlay of the UV light upon the surface or area to be sanitized. The UV light pod 10 is photometrically designed to increase the efficiency and efficacy of disinfection over time with a 360 degree overlap of UV light beam angles. The UV light pod 10 illustrated in FIG. 7 includes six UV light panels 100, referred to individually as 100A, 100B, 100C, 100D, 100E, and 100F, secured to six portions of the support structures 12, referred to individually as support structures surfaces 12A, 12B, 12C, 12D, 12E, and 12F, arranged to direct UV light in a direction to a surface which requires UV light exposure. Each of the six UV light panels individually as 100A, 100B, 100C, 100D, 100E, and 100F provide a UV light exposure range. For UV light panel 100A, the UV light is illustrated as lines 1000A-1000A. For UV light panel 100B, the UV light is illustrated as lines 1000B-1000B. For UV light panel 100C, the UV light is illustrated as lines 1000C-1000C. For UV light panel 100D, the UV light is illustrated as lines 1000D-1000D. For UV light panel 100E, the UV light is illustrated as lines 1000E-1000E. For UV light panel 100F, the UV light is illustrated as lines 1000F-1000F. Where the UV light lines cross, the area includes overlapping UV light exposure. As shown in FIG. 7, the UV light pod 10 provides 360 degree UV light exposure and overlap of UV light beam angles. Such arrangement provides for increased efficiency and efficacy of disinfection over time.

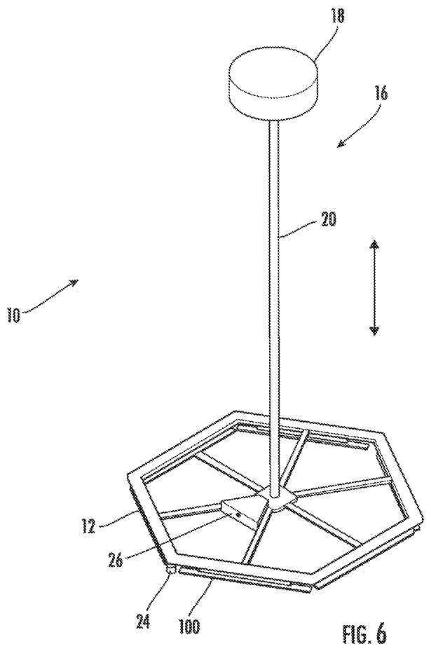

FIG. 5 illustrates the UV light pod 10 secured to a celling 14. The UV light pod 10 may be hung from the ceiling 14 at a fixed position, using for example a rod or secured to a beam. Alternatively, the UV light pod 10 may be designed as a dynamic unit so that the support structure 12 can be lowered towards the surface/area or raised up towards the ceiling 14, away from the surface or area to be exposed to the UV light. Referring to FIG. 6, the UV light pod 10 may be a lift or hoist system 16 configured to move and place the support structure 12 at one or more positions relative to the ceiling 14. The lift or hoist system 16 may include a ribbon lift or hoist including an actuator 18 configured to drive a ribbon or post/rod 20 up/down. The ribbon or post/rod 20 may be secured (via mechanical fasteners such as screws or bolts) to the support structure 12 at the support structure post receiving member 22, see FIG. 1.

To prevent accidental UV light exposure, the UV light pod 10 may include an in-use warning system. The in-use warning system may include an LED beacon in-use warning light 24, see FIG. 3. The LED beacon in-use warning light 24 is operatively connected to the UV light panels 100. Accordingly, when the UV light pod 10 is exposing the air, surfaces, or other parts of an area, the LED beacon in-use warning light 24 is turned on automatically. As soon as the UV light is turned off, the LED beacon in-use warning light 24 automatically turns off. The LED beacon in-use warning light 24 may be configured to flash and have an audio alarm. The beacon in-use warning light 24 preferably has a separate power cord (not shown) to be plugged into a separate electrical outlet (not shown). To further prevent accidental UV light exposure, the UV light pod 10 may also include an occupancy safety system. The occupancy safety system may be configured to either prevent the UV light panels 100 from operating (if in the “OFF” operation position) or stopping the operation of the UV light panels 100 (if in the “ON” operation position) if motion is sensed.

The UV light pod 10 may comprise a box 26. The box 26 may include all the hardware necessary to operate and control the UV light pod 10 functionality; particularly the electrical functioning of the UV light pod 10, and may include, for example, wiring for each of the UV light panels 100 or the LED beacon in-use warning light 24, fuses, terminal block, transformers, and the like. Any cables used may be connected internally with din rails, and be passed through metal grommets out to each UV light panel 100. The box 26 may also contain one or more structures or electrical components, such as control panels or circuit boards, processors, memory, electrical outlet receptacles, power cord receptacles, timer units, fuses, terminal blocks, transformers, or WIFI switches, such as a SONOFF WIFI connector, to be used to turn all UV light panels 100 “ON/OFF” via an app, wireless technology components such as WIFI/RF/BLUETOOTH transmitters and/or receivers, electrical connectors, and other hardware or software, operatively connected to one or more structures (i.e. UV lights, sensors, timers) to drive functionality to operate the UV light pod 10, including allowing the user to turn on/off UV lights, turn off audio or visual alarms, or program on/off times or cycles. The box 26 may also include a WIFI switch (for wireless operations) with power cord (not shown), such as a SONOFF WIFI connector, to be used to turn all UV light panels 100 “ON/OFF” via an app.

Alternatively, the UV light pod 10 may be RF (radio frequency) compatible and include an RF key fob for quick on/off functioning. The UV light pod 10 may be configured to operate using other wireless technologies as well, such as BLUETOOTH. The UV light pod 10 may be configured to be a smart device, and include the necessary hardware and/or software for such operation and functionality. The UV light pod 10 may be configured to communicate with an electronic device, such as a smart phone or computer tablet, i.e. IPAD. Control of the UV light functioning, i.e. on/off, scheduling of on/off times, run times, and when each (or all) of the individual UV light panels 100 turns on/off may also be accomplished using a cell phone or other remote electronic device.

Any function of the UV light pod 10 may also be controlled by a separate, hard wired or wirelessly connected, control panel located off of or away from the UV light pod 10 location. For example, in the case the UV light pod 10 is hung from a structure above the ground, the UV light pod 10 may be controlled by a control panel located on a separate wall easily accessible, similar to light switches that operate lights hung on the ceiling of a home.

The UV light pod 10 may include one or more occupancy sensor(s) 23, see FIG. 1, secured to any section or component of the UV light pod 10, including the support structure 12, internal beams 25, or to a light panel 100, such as motion sensors or heat/thermal sensors, within the sterilization range (air or surface, or area being exposed to the UV light) configured to immediately shut off the UV light pod 10 if a person or animal is within the sanitization range (UV light exposure) while the UV light pod 10 is in the “ON” or operating position, thus preventing accidental human/animal UV light exposure when the UV lights are operating. In one embodiment, each of the one or more UV light panels 100 may include its own occupancy sensors 23. All of the one or more UV light panels 100 may be turned off simultaneously, or each individual one or more UV light panels 100 may be turned off independently relative to each other.

To aid in the disinfection process, the UV light pod may include one or more ionizer(s) 27, such as the D5 Sterionizer manufactured by Eagle X Pro, see FIG. 1. Preferably, the ionizer 27 is designed to pump ions, such as 10 billion ions per second, into the UV light pod 10 without producing ozone. The positive and negative ions attach to microbes, such as virus/bacteria, breaking them down and rendering them unable to infect. The ionizer 27 may be placed and secured to any portion of the UV light pod 10.

The UV light pod 10 may also include a removable cover 28. The removable cover 28 may be used to allow one or more types of indicia, such as words, letters, phrases, symbols, pictures, lighting, or other advertising type materials, to be placed thereon.

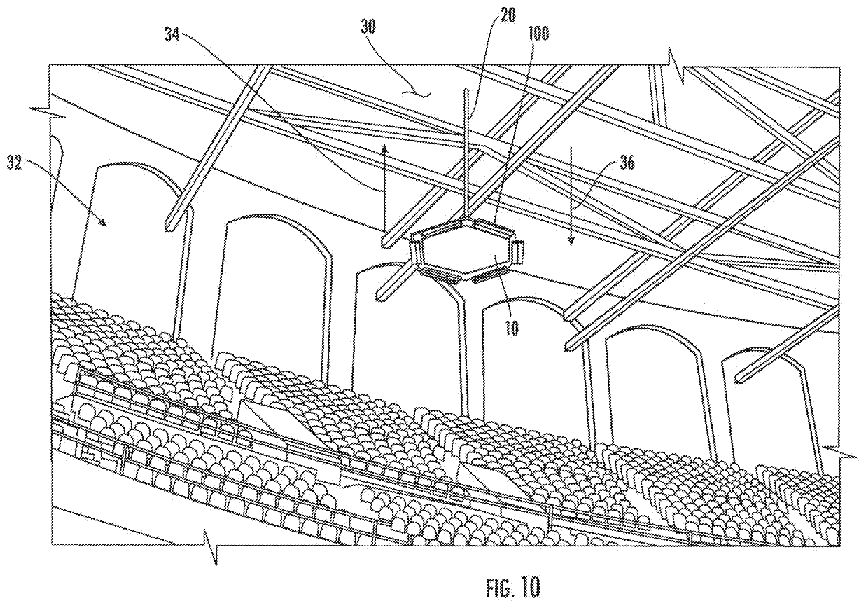

FIG. 10 illustrates the UV light pod 10 secured to a ceiling 30 of a stadium 32 by the ribbon/rod 20. In this arrangement, the UV light pod 10 hangs from a structure (i.e. ceiling 30) so that the light panels 100 are arranged, positioned or orientated in a position that is parallel relative to the stadium floor. If the UV light pod 10 includes the lift system, the UV light pod 10 may be moved up, see arrow 34 or down, see arrow 36, in order to adjust the height or positioning of the UV light pod 10.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary, and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A UV light based sanitization pod comprising:
a support structure having multiple surfaces arranged to direct UV light in a direction which is parallel to a ground surface;
a plurality of UV light panels having a UV light source configured to disinfect a surface or air.

2. The UV light based sanitization pod according to claim 1, further including a rod configured to suspend said UV light based sanitization pod above a ground surface.

3. The UV light based sanitization pod according to claim 1, further including a lift system configured to move said support structure to different positions relative to the surface for which UV light based sanitization is required.

4. The UV light based sanitization pod according to claim 3, wherein said lift system comprises a ribbon lift or post attached to said support frame; and an actuator configured to drive said ribbon or post in an upward or downward direction to the surface for which UV light based sanitization is required.

5. The UV light based sanitization pod according to claim 1, wherein said support structure has a hexagonal shape.

6. The UV light based sanitization pod according to claim 5, wherein said support structure has six UV light panels arranged to provide 360 degree overlap of UV light exposure.

7. The UV light based sanitization pod according to claim 1 wherein each of said light panels are removably secured to said support structure.

8. The UV light based sanitization pod according to claim 1, wherein said UV light panels produce UV-C light.

9. The UV light based sanitization pod according to claim 1, wherein said UV light panels produce UV-C light in the range of 200 nm-400 nm.

10. The UV light based sanitization pod according to claim 9, wherein said UV light panels produce UV-C light at about 254 nm.

11. The UV light based sanitization pod according to claim 1, further including a visual warning device.

12. The UV light based sanitization pod according to claim 1, further including an audio safety warning device.

13. The UV light based sanitization pod according to claim 1, further including one or more occupancy sensors configured to immediately shut off said UV light panels if a person or animal is within a disinfection range while said UV light based sanitization pod is in operating position.

14. The UV light based sanitization pod according to claim 1, wherein said UV light based sanitization pod is configured to operate via wireless technology.

15. The UV light based sanitization pod according to claim 14, wherein said wireless technology is WiFi, RF, or Bluetooth.

16. The UV light based sanitization pod according to claim 1, wherein said UV light based sanitization pod is configured to communicate with a smart phone or a computer tablet.

17. The UV light based sanitization pod according to claim 1, further including an ionizer.
